(19) United States
(12) Patent Application Publication
(10) Pub. No.: US 2021/0378916 A1
(43) Pub. Date: Dec. 9, 2021

(54) PERSONALIZED DRUG DOSE DISPENSER AND METHOD

(71) Applicant: SHALON VENTURES RESEARCH LLC, Palo Alto, CA (US)

(72) Inventor: Tadmor SHALON, Palo Alto, CA (US)

(73) Assignee: SHALON VENTURES RESEARCH LLC, Palo Alto, CA (US)

(21) Appl. No.: 17/283,602

(22) PCT Filed: Oct. 17, 2019
(86) PCT No.: PCT/US2019/056650

§ 371 Date: Apr. 8, 2021

Related U.S. Application Data
(60) Provisional application No. 62/748,072, filed on Oct. 19, 2018.

(51) Int. Cl. A61J 7/00; G16H 20/13

(52) U.S. Cl. CPC A61J 7/0053; A61J 2205/60; G16H 20/13

(57) ABSTRACT

A dispensing system including a handheld dispenser and a dispenser controller. The handheld dispenser may have a housing, a cartridge removably disposed within the housing and containing a dose form to be dispensed, the dose form with an active pharmaceutical ingredient (API), the cartridge having an internal piston movable within a reservoir containing the dose form and a port through a distal tip in communication with the reservoir, the distal tip being sized and configured to be inserted into the user's mouth. A motor disposed in the housing moves the piston to dispense a volume of the dose form from the cartridge through the port. The dispenser controller adjusts the volume of the dose form dispensed by the dispenser based on information about effectiveness of the API in treating a condition of the user.

### CROSS REFERENCE TO RELATED APPLICATIONS

This application claims the benefit of U.S. Application No. 62/748,072, filed Oct. 19, 2018, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

The present invention relates to a drug dispenser to minimize drug intake by tailoring each dose to the user's response to the medication and stimulating their placebo response. Minimizing the exposure to the drug reduces the user's tolerance and undesired side effects thus extending the drug's therapeutic benefits. Among several drugs which lend themselves to smart personalized dispensing are drugs with narrow therapeutic indices, dose dependent side effects and efficacy based on patient parameters such as weight or pre-existing conditions. As an example, the present invention describes use of the invention for, drug to aid in initiating and maintaining sleep.

Most adults experience problems falling to sleep either in the beginning of the night or after waking up in the middle of the night. In a recent survey of over 1,000 US consumers, over 50% reported having trouble falling and staying asleep at least several times a week and over 30% reported using sleeping aids at least several times a week. A significant majority indicated that they want a safe, non-habit-forming solution to this problem. Most active pharmaceutical ingredients (“APIs”) available with or without prescription are taken orally as pills, capsules, gel caps or a cup of liquid. Yet while users desire immediate relief from insomnia, the active ingredients in these dose forms must be absorbed through the gastrointestinal tract and subject to first pass metabolism by the liver before becoming active systemically. Hence, these APIs require a long time and higher doses to achieve sufficient blood concentration to induce sleep. Correspondingly, such doses have a longer half-life in the user's blood stream inducing undesirable tolerance requiring larger doses to attain the same therapeutic effect and increasing side effects such as drowsiness when the user has to wake up. These side effects are exacerbated when the API is taken in the middle of the night, and in many cases the FDA requires vendors to warn the users not to take the API in the middle of the night or not to drive or operate machinery in the morning if they do.

### SUMMARY OF THE DISCLOSURE

It is well known that psychological factors such as anxiety about being able to fall asleep exacerbates insomnia. Hence dose forms that stimulate psychological factors such as the placebo effect enhance the API's therapeutic index. Immediate sensations such as taste, color, mode of administration can stimulate and increase the API's therapeutic effects.

In addition, to initiate or maintain sleep, each user requires a different minimum dose that depends on many factors such as recent sleep history, body weight, recent responsiveness to the API, time of night, and other environmental condition under which it is administered. Beneficial placebo effects can be further stimulated if the user doesn't know how much API they are receiving, and personalization can be automatically implemented by the system utilizing such knowledge.

Unlike other placebo interventions used in medical practice where placebo pills are substituted for, or alternated with, pills containing an API, the device and method of this invention can titrate the placebo effect and maximize it. It is capable of modulating the doses while masking them from the user in order to enhance and prolong the placebo effect.

The present invention relates to a smart dispenser configured to conveniently dispense an API in various doses, including a placebo or sub-therapeutic dose, in a manner responsive to each user's unique physiology and circumstances in order to increase the API's therapeutic index (i.e., to optimize efficacy while minimizing side effects). The smart dispenser enhances the placebo effect by various means including masking the actual delivered dose. The smart dispenser interacts wirelessly with a smartphone or other devices that measure the user's response to the API by objective physiological measurements or self-reporting, and it adjusts the dosing regimen to minimize the user's exposure to the API while optimizing its therapeutic index. The smart dispenser also enhances API safety by limiting the total delivered dose, the timing of doses, and preventing children or others for accessing the API contained in the dispenser.

In particular, the present invention dispenses APIs to assist in initiation or maintenance of a sleep cycle in response to the user's schedule, sensitivities as derived from user response to previous API administrations with the goal of minimizing API exposure, possibility for over dosing, side effects and API tolerance.

The device and API combination utilize a user interface, such as a smartphone, where the user's desired sleep schedule and wake up time is already captured and devices operating in conjunction with the smartphone or independently that capture the user's sleep patterns in order to provide feedback to the algorithm that titrates the optimal dose in part based on the user's past physiological sleep responses.

One aspect of the invention provides a dispensing system having a handheld dispenser and a dispenser controller, with the handheld dispenser having a housing; a cartridge removably disposed within the housing and containing a dose form to be dispensed, the dose form comprising an active pharmaceutical ingredient (API) (for example, a sleep aid such as, e.g., diphenhydramine), the cartridge having an internal piston movable within a reservoir containing the dose form and a port through a distal tip in communication with the reservoir, the distal tip being sized and configured to be inserted into the user's mouth; a motor disposed in the housing; a movable shaft operably connected to the motor and to the piston; an actuator operably connected to a motor controller and adapted to be actuated by the user to cause the motor to dispense a volume of the dose form from the cartridge through the port, with the dispenser controller being adapted to adjust the volume of the dose form dispensed by the dispenser based on information about effectiveness of the API in treating a condition of the user.

In some embodiments, the handheld dispenser also has a wireless transmitter/receiver adapted to communicate with an associated smartphone. In such embodiments, the dispensing system may be configured to receive dispensed volume control information from the smartphone based on information obtained by the smartphone about effectiveness of the API in treating the user. For example, if the API is a sleep aid, the information may include information about the quality of the user's sleep, the length of the user's sleep, and/or the user's feeling of restedness.

In any of these embodiments, the dose form may include a detectable ingredient such as a flavoring agent (e.g., menthol) and/or a numbing agent.

In any of these embodiments, the cartridge may have a reservoir containing the dose form, with the distal tip communicating with the reservoir. In such embodiments, the dispenser may also have a window through which contents of the reservoir can be viewed. The cartridge may also have a valve in the tip adapted to open to deliver the volume of dose form from the reservoir through the port. The valve may have a valve element extending from a stretchable shaft, the stretchable shaft adapted to permit the valve element to move away from a valve seat when movement of the piston within the reservoir increases pressure within the reservoir.

In any of these embodiments, the system may also have a removable cap adapted to cover the distal tip. In any of these embodiments, the system may also have a removable cover adapted to permit insertion and removal of the cartridge to and from the handheld dispenser.

In embodiments of the invention, the system has at least two cartridges each containing different dose forms. In such embodiments, cartridge is a first cartridge and the dose form is a first dose form, the dispensing system further comprising a second cartridge containing a second dose form different than the first dose form. In some such embodiments, the second cartridge holds its dose form in a reservoir that is in communication with the port through the distal tip.

Another aspect of the invention provides a method of administering an active pharmaceutical ingredient (API) (for example, a sleep aid such as, e.g., diphenhydramine) from a dispensing system to a user to treat a condition of the user. Some embodiments of the invention include the steps of actuating a user input of the dispensing system at a first time; in response to actuation of the user input at the first time, dispensing a first volume of a dose form into the user's oral cavity, the dose form comprising the API and a detectable ingredient; obtaining information regarding effectiveness of the dispensing of the first volume in treating the condition; and actuating the user input of the dispensing system a second time after the first time to dispense a second volume of the dose form into the user's oral cavity, the second volume being automatically controlled by the dispensing system to differ from the first volume based on the information.

Where the condition being treated is the user's sleeplessness, the information can include information about the quality of the user's sleep, the length of the user's sleep, and/or the user's feeling of restedness.

In such embodiments, the detectable ingredient may be a flavoring agent (e.g., menthol) and/or a numbing agent.

In any of these embodiments, the second volume may be less than the first volume. In such embodiments, the second volume may have a sub-therapeutic dose of the API. Alternatively, in any of these embodiments, the second volume may be more than the first volume. In embodiments in which the API is a sleep aid, method may include the step of obtaining information about expected hours of remaining sleep time, the second volume being automatically controlled by the dispensing system to differ from the first volume based on the expected hours of remaining sleep time.

In any of these embodiments, the method may also include, before dispensing the second volume, the step of determining whether the total dose delivered in the first volume is less than a predetermined maximum dose and, if not, providing an indication to the user that the second volume of the dose form will not be delivered. In any of these embodiments, the method may also include, before dispensing the first volume, the step of determining whether the user is an authorized user.

In any of these embodiments, the dispensing system may include a handheld dispenser. In such embodiments, the dispensing system may also have a replaceable cartridge within the handheld dispenser containing the dose form. The cartridge may have a dispensing tip configured to be inserted into the user's mouth, with the method including the further step of inserting the dispensing tip into the user's mouth prior to dispensing the first volume or the second volume. The dispenser may also have a window through which contents of the cartridge can be viewed.

In embodiments of the invention, the system has at least two cartridges each containing different dose forms. In such embodiments, cartridge is a first cartridge and the dose form is a first dose form, the dispensing system further comprising a second cartridge containing a second dose form different than the first dose form. In some such embodiments, the second cartridge holds its dose form in a reservoir that is in communication with the port through the distal tip, and the method may include the step of inserting the dispensing tip into the user's mouth prior to dispensing the first volume or the second volume.

In any of these embodiments, the dispensing system may also have a smartphone running an application, the method further comprising entering the information through the application on the smartphone. In such embodiments, the method may include the step of setting the second volume with the application on the smartphone and communicating information about the second volume from the smartphone to the dispenser.

In any of these embodiments, the API dose in the first volume may be substantially absorbed by oral mucosa of the user. In any of these embodiments, the first volume may be small enough to avoid stimulation of a swallowing instinct of the user. In any of these embodiments, the dose form may includes a penetration enhancer and/or a viscosity modifier.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a side cross-sectional view of a dispenser according to an embodiment of the invention.

FIG. 2 is an exploded view of the embodiment of FIG. 1.

FIG. 3 is a side cross-sectional view of an embodiment of a cartridge for use with the dispenser of FIG. 1.

FIG. 4 is a perspective view of a dispensing system according to an embodiment of the invention.

FIG. 5 is a side cross-sectional view of a dispenser according to another embodiment of the invention.

FIG. 6 is a perspective view of components of a dual cartridge dispenser according to another embodiment of the invention with some elements illustrated transparently to show internal elements.

FIG. 7 is a flow chart illustrating a typical automatic dose adjustments based on whether the therapeutic goals have been achieved and side effects minimized.

### DETAILED DESCRIPTION

In some embodiments, in response to the user's request the device dispenses a volume of an optimal API dose based on the user's sleep responses to previous doses, sleep time left in the user's sleep schedule, total dose previously delivered in 24 hours, and other user parameters. The API dose is delivered through an appropriate vehicle such as a liquid or a gel (“dose form”) delivered through a reusable or disposable oral applicator. The dose form (e.g., the API dose plus any excipients) is delivered to the oral and esophageal mucosa (“oral mucosa”) where it can be absorbed without being degraded, subjected to variable absorption rate, affecting the user's GI tract, or encountering delayed plasma PK and hepatic first pass metabolism. The dose form may contain a flavoring agent to mask or enhance the taste of the underlying API and/or other components of the dose form. The flavoring agent may stimulate a placebo response even when the dose form contains only a sub-therapeutic amount of API, or no API. For example, diphenhydramine has a bitter taste and it is a topical numbing agent. Menthol can be added to the formulation to amplify the taste beyond the amount needed to mask the bitterness of the drug, thereby masking both the presence and the reduction or absence of the drug in a delivered volume of dose form, and a numbing agent may be added to simulate the numbing effect of diphenhydramine to mask the drug's reduction or absence in a delivered volume of dose form.

In some embodiments, the dispenser of this invention may be connected to the user's smartphone via cable or RF link such as Bluetooth (“BLE”) or WiFi. The smartphone (or other connected computing device) calculates the appropriate dose required based on the user's past history of requests, their self-rated efficacy of previous doses, sleep monitoring devices reporting on the user's sleep patterns and response to previous doses, the time of the night when the request is made, the API's PK profile, and/or the user's desired waking time.

In some embodiments, the dose form is an active ingredient or an API such as diphenhydramine dispersed in a liquid or gel. In some embodiments, the dose form has a volume small enough to avoid stimulation of the swallowing instinct. In some embodiments, even if swallowed, the dose form has a volume small enough such that most of the absorption of the API will rapidly take place before the dose form reaches the user's stomach. In some embodiments, the API dose may be delivered in multiple discrete volumes. The rapid PK of a smaller API dose quickly induces sleep and subsequently quickly clears from the blood stream to avoid drowsiness or other side effects when the user wakes up and minimizes systemic exposure to the API over prolonged period that could induce API tolerance.

In some embodiments, the dose form may contain flavors to mask or enhance the taste of the active ingredient, muco-adhesive agents (such as, e.g., polyacrylates, hydrogels, ethylene vinyl alcohol, polyacrylates, polycarbophil, chitosan) to increase its residence time in the mouth, penetration enhancers (such as, e.g., sodium lauryl sulphate, bile acids and derivatives, poly-L-arginine) to accelerate absorption, viscosity modifiers, gelling ingredients including thixotropic agents (such as, e.g., fumed silica or colloidal silica) to titrate how much of the formulation will be absorbed retained and absorbed in the oral mucosa and how much if any will flow to the stomach and be absorbed in the GI tract.

Table 1 lists several formulations for an active ingredient and excipients according to embodiments of the invention. The amount of solvents can be adjusted to determine how much of the API dose will be absorbed orally or in the GI tract. A sweetener (e.g., Splenda®) and/or coloring or flavoring agent may be added to any of these formulations to make them tolerable yet distinctive in taste and appearance to stimulate the placebo effect. For example, embodiments of the dispenser may have two (or more) reservoirs or cartridges, one containing the API dose form and one containing the other formulation components (including the flavoring component) but without the API.

TABLE 1

| | | | Compound amount mg/g | | | | | |
|---|---|---|---|---|---|---|---|---|
| | | | Formula | Formula | Formula | Formula | Formula | Formula |
| ## | Component | Purpose | 1 | 2 | 3 | 4 | 5 | 6 |
| 1 | Diphenhydramine | Active | 100 | 100 | 100 | 100 | 100 | 100 |
| | hydrochloride | ingredient | | | | | | |
| | | (API) | | | | | | |
| 2 | Ethyl alcohol | Solvent for | 200 | n/a | 200 | n/a | n/a | n/a |
| | | API | | | | | | |
| 3 | Propylene glycol | Solvent for | n/a | 50 | n/a | 50 | 50 | 50 |
| | | API | | | | | | |
| 4 | Deoxyglycocholate | API | n/a | n/a | n/a | 50 | n/a | 50 |
| | sodium | Penetration | | | | | | |
| | | enhancer | | | | | | |
| 5 | Deoxitaurocholate | API | n/a | n/a | n/a | n/a | 50 | n/a |
| | sodium | Penetration | | | | | | |
| | | enhancer | | | | | | |
| 6 | Dodecyl 2-N,N- | API | n/a | n/a | 50 | n/a | n/a | n/a |
| | dimethyl- | Penetration | | | | | | |
| | aminopropionate | enhancer | | | | | | |
| | hydrochloride | | | | | | | |
| 7 | Dextran 250,000 | Viscosity | n/a | 25 | n/a | n/a | n/a | n/a |
| | | modifier | | | | | | |
| 8 | Carbomer 940N | Viscosity | n/a | n/a | n/a | n/a | n/a | 5 |
| | | modifier | | | | | | |
| 9 | Water | Solvent for | 700 | 825 | 650 | 800 | 800 | 795 |
| | | API | | | | | | |

The device dispenses variable volumes of the dose form based on the calculated desired dose. For example, if the dose form has a concentration of 1 mg/5 μL, a 10 mg API dose may consist of a 50 μL of the dose form dispensed to the user's mouth via a thin tip of the disposable cartridge. If the user requests a dose when there are only, e.g., 2 hours left to sleep on their schedule, the device will dispense a much smaller dose, a sub-therapeutic dose, or a placebo dose (i.e., a dose form with no API or active ingredient, only a flavoring agent and/or numbing agent) from an adjacent independently controlled reservoir in the system.

In some embodiments, the smart dispenser stores key variables in its local memory and has a real-time clock circuit. These variables and real-time are updated periodically via BLE from an associated smartphone. For example, for the sleep application, the variables such as maximum API dose in 24 hours, minimum effective API dose, starting API dose, secondary API dose if requested in middle of the night, dose already dispensed in the past 24 hours, wake up time (derived from alarm clock or calendar stored on or accessed by the smartphone), status of a child safety feature requiring the dispenser to be within BLE range of the smartphone to dispense, etc. The smart dispenser logs every event and dose and when it is within BLE range transmits the time stamped event log to the smartphone which in turn sends it up to a remote server for storage and processing. The maximum amounts of API and frequency of administration can be controlled by the algorithm and instructed by a healthcare professional via the remote server or smartphone, and stored in the smart dispenser. Alternatively, the smart dispenser or the phone can run a learning algorithm by recording the user's response to each dose via a simple query, for example, how long did it take you to fall asleep after you took a dose? How refreshed do you feel in the morning? If the latency of sleep onset is long, the API dose can be automatically increased by the remote server, smartphone or smart dispenser (“Processing Unit”) until the preprogrammed maximum safe dose limit is reached. If the user is not feeling refreshed in the morning compared to days when drug was not administered, the next dose can be reduced automatically from the latest dose administered (initial dose or the middle of the night dose). The middle of the night dose can be automatically adjusted from a maximum level depending on how many more hours the user has to sleep as estimated by the smart dispenser or the smartphone and communicated via BLE to the smart dispenser. As described elsewhere, objective criteria for these subjective measures can be obtained and used to drive the algorithm modifying the doses in a similar manner. In addition, the Processing Unit can periodically decrease the doses, while keeping it above a minimum dose parameter, to see whether a lower dose will result in a acceptable therapeutic effect and thus automatically reduce the user's exposure to drug and its side effects.

The minimum and maximum volume of delivered dose form can vary by a factor of ten or more. Therefore, in order to mask differences in the delivered volume, during delivery of a smaller dose from, the dispense cycle can be extended by slowing down the gear motor driving the dispenser, extending the tactile feedback of the motor by vibrating it back and forth, and extending the period in which the blinking LED is displayed to mask the actual dose delivered in order to enhance the placebo effect of each dispensing cycle. The resulting doses required to induce sleep are smaller than the typical adult oral dose of 50 mg (ZZZQuil® and FDA monograph for diphenhydramine) or other doses delivered via capsules or tablets or gelcaps (e.g., Benadryl®, Sominex®, Unisom®, Advil PM®, Tylenol PM®)). This is accomplished because of the rapid oral mucosa absorption and the stimulation of the placebo effect by means described above.

In some embodiments, the small portable bed-side device dispenses the required dose within a few seconds when the user presses a button so that minimal sleep disruption occurs during the application while insuring the user's safety by automatically limiting the total API delivered in multiple applications over 24 hours.

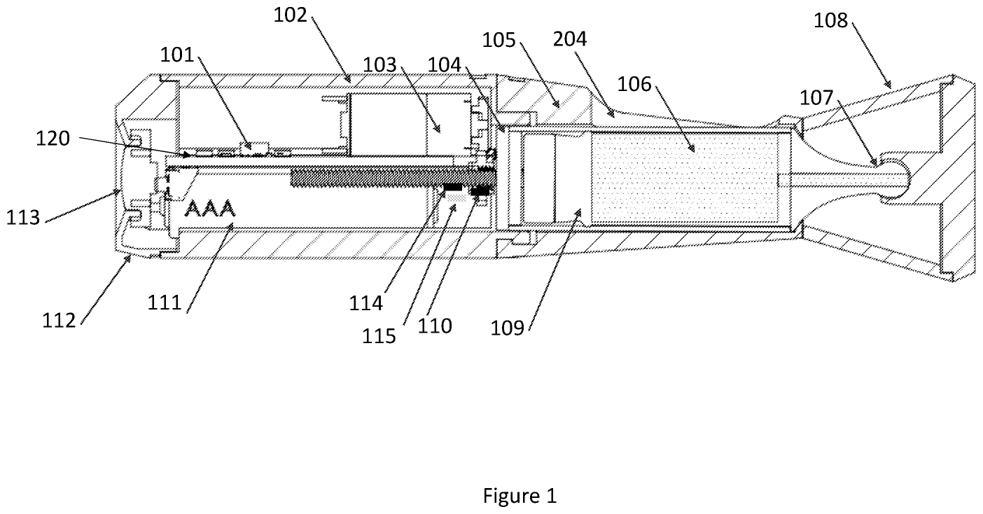

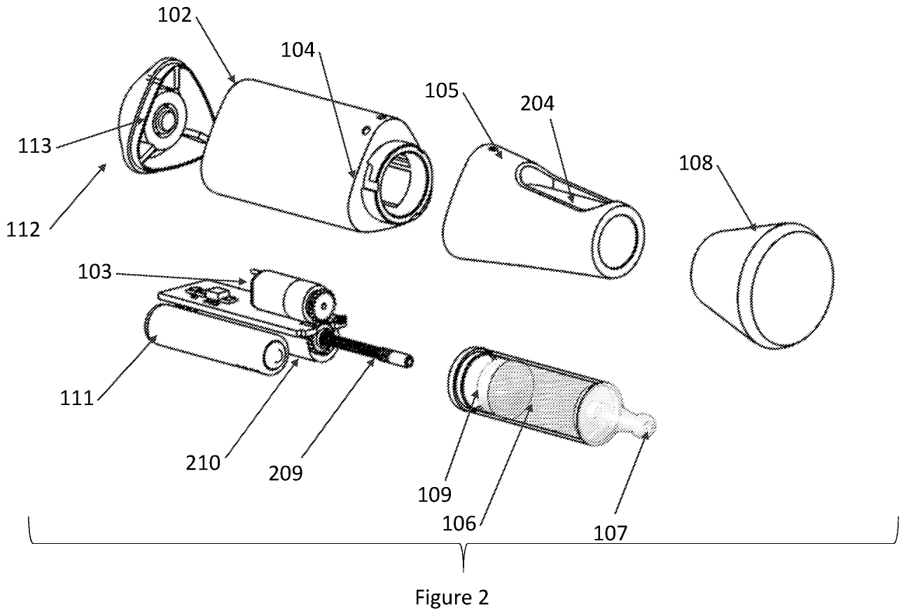

FIGS. 1 and 2 show one embodiment of a dispenser 100 according to an aspect of the invention. Dispenser 100 holds a disposable cartridge 106 (seen through a window 204) containing the dose form including the API which is to be ejected in small amounts via a port in the cartridge tip 107 positioned in the user's mouth. The dosing process is initiated by the user activating switch 113, which causes the microcontroller 101 to activate a gear motor 103 while monitoring shaft encoder 114 as it turns a threaded gear 210 to drive a threaded rod 209 pushing forward a piston assembly 109 within cartridge 106 to eject a precise amount of dose form through an opening in cartridge tip 107 to the user's oral mucosa. A removable cartridge retention cover 105 may be rotated to rotate bayonet retention pins 104 in slots in dispenser housing 102 to lock and unlock the cover 105. Removing the cover 105 activates a switch 110 that triggers the microcontroller 101 to withdraw the threaded rod 109 so that a used cartridge may be removed. When a new cartridge is inserted, the switch 110 is triggered and the motor is driven forward until the microcontroller detects an increase in drive current due to the increased torque caused by the threaded rod contacting the piston assembly of the new cartridge. The device is powered by batteries 111 (e.g., 2 AAA batteries) that can be replaced by removing battery cover 112. The microcontroller can keep track of the doses dispensed and via LED 115 inform the user when the dose has been dispensed, when the cartridge is running low, or when batteries need to be replaced. The cartridge tip cover 108 snaps over cartridge tip 107 and can also serve as a dispenser stand to reduce space required on a night stand. The electronics may include an optional wireless transmitter/receiver 120 to communicate with, e.g., an associated application running on the user's smartphone (not shown).

The microcontroller also contains BLE electronics that automatically pair with the user's phone and communicate to the cloud connected app for logging dispensing events while receiving updated dosing parameters that can be executed without smartphone connectivity.

In some embodiments, cartridge 106 is clear or translucent, and a window 204 allows a user to visualize the amount of dose form left in the cartridge 106. In another embodiment, the gear 210 contains a clutch activated by the cartridge so that when the cartridge 106 is not fully captured by retention housing 105 the threaded rod 109 is free to move back allowing the new full cartridge to push the rod back. When the cartridge is fully inserted, it activates the threaded clutch to engage the threaded rod and push it forward when activated by gear motor 103.

In an alternative embodiment, a micropump (e.g., Takasago® RP-Q1) connected to the reservoir can pump the dose form to a tip under similar computer control.

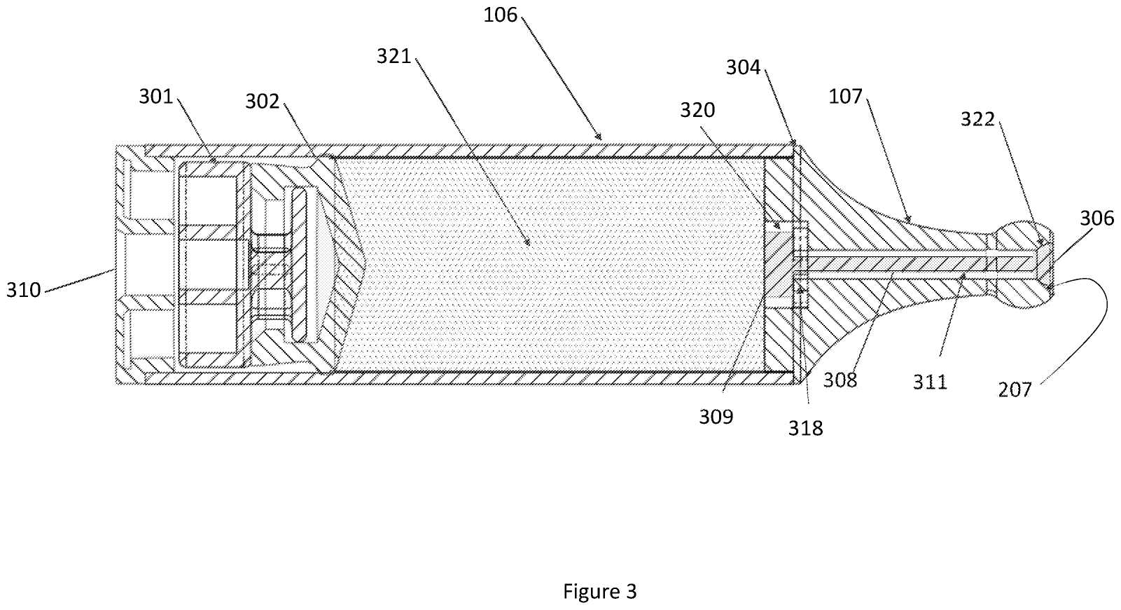

FIG. 3 shows additional details of cartridge 106 from the embodiment of FIGS. 1 and 2. Because of the small volumes dispensed, in some embodiments the dispenser cartridge's tip may contain a one-way valve ensuring that the solution in the cartridge does not evaporate even after prolonged storage and that the API density within the solution remains constant. The valve also protects the API solution within the cartridge from microbial contamination in the oral cavity by providing a barrier to diffusion or aspiration of saliva into the cartridge and thus reducing or eliminating the need for a preservative. Movable piston 109 is disposed on a piston carrier 301. A press-fit cap 310 is disposed in the proximal end of cartridge 106 after filling it with dose form. A valve element 306 is disposed in an opening 207 at the distal end of the cartridge tip channel 311 extending from an opening 320 at the distal end of cartridge reservoir 321 to the cartridge tip opening 207. When not dispensing a volume of dose form, a proximal surface of valve element 306 rests against a complementarily shaped valve seat 322 to seal the distal end of channel 311. In this embodiment, valve element 306 is shaped as a hemisphere. A stretchable bar 308 (formed, e.g., from an elastomeric material) extends proximally from valve element 306 to a valve anchor 309 disposed in opening 320 proximal to the proximal end of channel 311. The diameter of anchor 309 is smaller than the diameter of opening 320 (leaving an annular passage around anchor 309), and the diameter of bar 308 is smaller than the diameter of channel 311, thus defining an annular passage around bar 308 through channel 311. One or more spacers 318 supports anchor away from the proximal opening of annular channel 311 to permit movement of the dose form (or other contents of reservoir 321) past anchor 309 to channel 311. When the dispenser's motor advances piston 109 in cartridge reservoir 321, the dose form inside of cartridge reservoir 321 is expelled through the annular opening around anchor 309 into channel 311. The pressure of the dose form in channel 311 pushes valve element 306 distally away from valve seat 322, thereby stretching bar 308 and opening an annular passage around valve element 306 and through opening 207. When the piston ceases moving and the desired volume of dose form has been delivered, the pressure inside of channel 311 drops, bar 308 returns to its original length, and valve element 306 seals opening 207.

Prior to insertion into the dispenser, the reservoir 321 of cartridge 106 may be filled using an automatic fast throughput filling machine that dispenses a precise amount of dose form into the cartridge through one end 304 of the cartridge. Thereafter, the tip 107 is pressed into end 304 of the cartridge. The tip 107 is designed to displace a precise amount of dose form when it is installed into a full cartridge during automatic high-speed filling. When the tip is fully installed, the displaced dose form precisely fills the dispensing channel 311 in order that the cartridge contains no air and is ready to dispense the first volume of dose form when installed on the smart dispenser.

In some embodiments, the cartridge tip 107 has a square channel, and the snap on cover 108 has a half round section that fits into the square channel to seal the tip to prevent evaporation of the dose form during prolonged storage and ensure that a desired volume of dose form with a fixed API concentration can be dispensed every time the user activates the smart dispenser. In another embodiment the tip contains an elastomeric insert 308 pressed into the tip's dispensing channel.

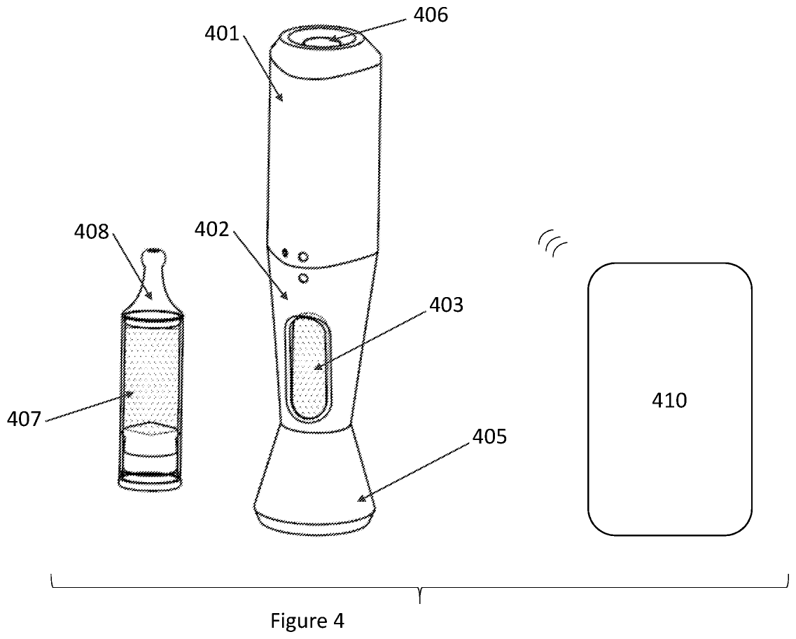

FIG. 4 shows an ergonomic embodiment of the dispenser 401 with an activation button 406, cartridge retention cover 402 and an installed cartridge shown through the window 403 standing on cap 405 next to a spare cartridge 407 with dispensing tip 408. FIG. 4 also shows a smartphone 410 communicating wirelessly with a transmitter/receiver (not shown) within dispenser 401. In some embodiments, information about effectiveness of an API (such as a sleep aid) may be entered by a user into an application running on smartphone 410, and smartphone 410 communicates dispensed volume control information to the microprocessor (not shown) in dispenser 401.

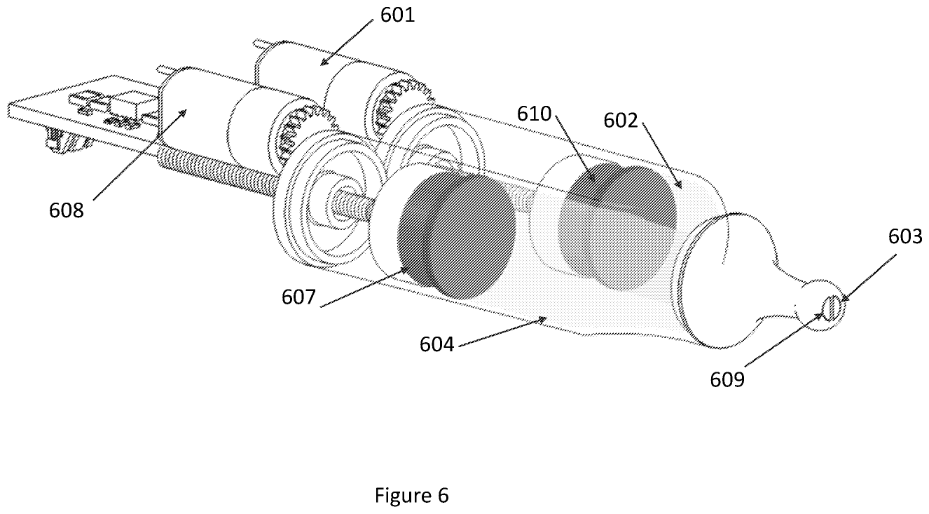

FIG. 6 shows a dispenser that may contain two or more disposable cartridges 602 and 604 each with its own movable piston 610 and 607, respectively, which are driven by motors 601 and 608, respectively, that are connected to the pistons by movable shafts, as in the earlier embodiment. The cartridges each have ports 603 and 609 in a common outlet tip. The dose form in cartridge 602 may include the API, and the dose form in cartridge 604 may include all ingredients of the dose form in cartridge 602 except the API. The multi-cartridge dispenser may contain one or more medicinal dose forms as well as a vehicle containing a liquid with no medication but with flavoring agent to act as a diluent to the medicinal dose forms or as a placebo dose. The window is eliminated from the cartridge retention cover so the user cannot tell from which reservoir the dose form is being dispensed in order to maximize the placebo response and minimize exposure to the drugs. For example, in a dual cartridge system, one cartridge may contain a narcotic or other pain medicine while the other a strongly flavored vehicle liquid. The system will dispense the vehicle with sufficient pain medication based on user's indication of pain and safe limits programmed by the prescribing physician with the goal of minimizing and eventually tapering off the pain medication while given the user the sensation that they are receiving pain medication and thus activating a strong placebo response. In yet another example, a three-cartridge system may contain sleep medication such as diphenhydramine formulation, the second cartridge may contain another active ingredient such as an NSAID, other pain relievers, melatonin or other sleep inducing or regulating agent, and the third cartridge may contain placebo flavored formulation to further dilute the API that can be dispensed simultaneously or alternatively to optimally induce and maintain sleep. For example, melatonin may be dispensed at the beginning of the night with diphenhydramine, while only diphenhydramine will be dispensed in the middle of the night.

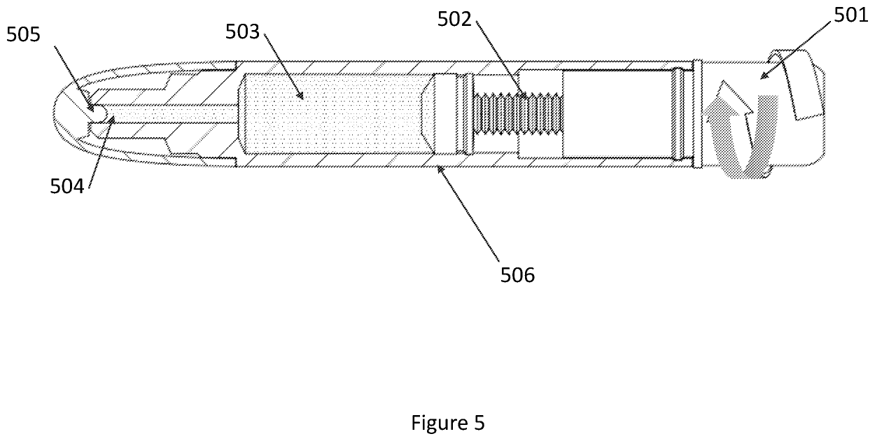

FIG. 5 describes a manual disposable dispenser where each full turn of the knob 501 on the back of the dispenser 502 dispenses a fixed volume of the dose form 503 to the user's mouth through its tip 504. A removable cap 505 may cover tip 504 to prevent evaporation of the dose form from the tip. This mechanism allows the knob 501 to turn in only the direction of moving the piston 506 forward. A turn of the knob delivering a single bolus of the dose form (e.g., 50 μl) may make a clicking sound. In use, the user may be advised to try a dose corresponding to 3-5 clicks of the knob 501 at the beginning of the night and an additional 1-3 clicks if the user awakes during the night, with a maximum of 10 clicks per 24 hour period. Tip 504 may be placed in the user's mouth, e.g., under the tongue, on the tongue or next to the cheek. The user may be advised to refrain from swallowing the dose in order to allow any API or active ingredient to be absorbed by the oral mucosa. In another embodiment, the knob is clicked like a tip of a ball point pen, with each click a single drop is dispensed from the reservoir described above.

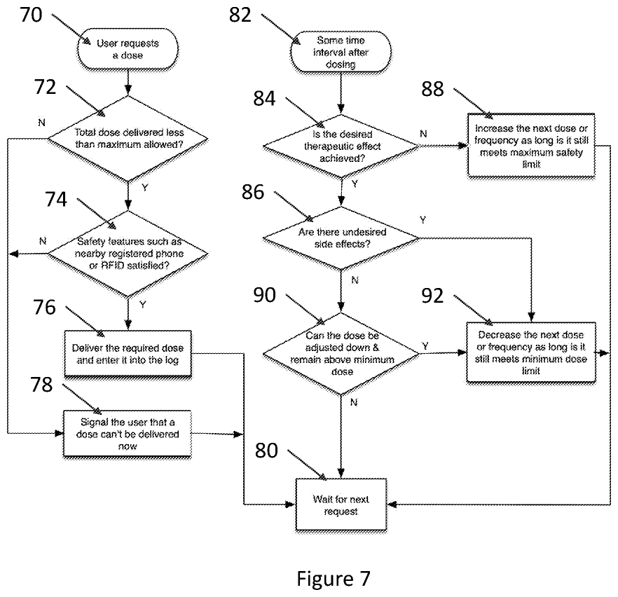

FIG. 7 shows a typical automatic adjustment of the dose regimen between maximum and minimum limits set into the dispenser via the connected smart phone which in turn receives this information from a remote server. The flow chart shows how the dispenser determines how to safely dispense a volume of the dose form each time the user activates it and how to adjust the dose to optimize the therapeutic effect while minimizing side effects and overall drug exposure based on user feedback or a relevant physiological measurement related to the dispenser directly, via the smart phone or via the remote server. The process starts with a user request 70 for a dose of the API. At block 72 the system (e.g., an application running on a smartphone connected to the dispense) determines if the total dose of the API delivered to the user during a relevant time frame is less than the maximum allowed. If so, at block 74 the system checks to see whether the request is legitimate by, e.g., confirming that the user is an authorized user by determining whether, e.g., the user has entered a password or other credentials into an associated smartphone or through the use of an RFID. If the safety features are satisfied, at block 76 the dispenser delivers a volume of the dose form to deliver the requested does of API and then waits for the next request 80. If the total dose of API is not less than the maximum allowed, or if the safety features are not satisfied, at block 78 the system signals the user to indicate that a dose cannot be delivered and then waits for the next request. In some embodiments the system also uses information about the therapeutic effect of an earlier dose to determine whether or how to deliver a subsequent dose. As indicated at blocks 82 and 84, some time interval after a dose the system considers user-entered efficacy information or automatically monitored information about the user to determine whether the desired therapeutic effect has been achieved. If so, and if there are not undesired side effects 86, the system at block 90 determines whether the dose can be adjusted down and remain above a minimum dose. If so, the system adjusts the dose down at block 92 and waits for the next dose request 80. If the desired therapeutic effect has not been achieved, the system increases the next dose at block 88, provided that the increased dose still meets a maximum safety limit, and waits for the next dose request 80. If the desired therapeutic effect has been achieved but there are undesired side effects, the system adjusts the dose down at block 92 for use at the next dose request 80.

In yet another aspect of the invention, the dose form is enclosed in a disposable cartridge that prevents a user from accessing the API without user authentication via their smartphone or similar registered device which has to be near the dispenser by sensing its BLE connection to ensure appropriate dosing, to prevent any overdosing, to make the system child proof, and to safeguard APIs such as opioids as required by law.

In yet another aspect of the device, the cartridge and smart dispenser can help enforce drug safety by ensuring that the only the patient who was prescribed the medicine is able to dispense it under dose control as described above. This is particularly helpful for drugs that could be abused such as opioids. In this embodiment, the cartridge contains an RFID or other serialized electronic ID. Each dispenser has a matching reader and will only dispense if the patient's personal smartphone or other registered electronic device capable of BLE communications is nearby in order to prevent unauthorized use of the drug. Each specific cartridge is tracked in the cloud to ensure that it has been dispensed appropriately by tracking the dosing for each dispenser so that if it runs out prematurely because a user or someone else adulterated the cartridge to extract some medicine without the dispenser, the smart dispenser will alert the healthcare prescriber via the cloud.

In yet another aspect of the invention, in order to elicit a placebo response and minimize API exposure, the amount of API dispensed can be masked from the user such that the user thinks they received an effective dose of API when they in fact received a sub-therapeutic dose, or no dose, of the API. For example, in embodiments in which the dispenser has a single cartridge containing an API dose form with a set concentration of API, in response to a request for a dose the dispenser can dispense a smaller volume of dose form than was delivered the prior time. If the overall volume is small, the user will not detect that the dispensed volume is smaller and will think that they received the same volume (and therefore the same API dose) as before. This belief can be reinforced if the dispenser provides dispensing cues that mask the reduced delivery volume, such as by running the motor longer than is necessary to dispense the reduced volume (e.g., slowing the motor operation) or by flashing delivery lights longer than is necessary to deliver the reduced volume. For dispenser embodiments having more than one dose form reservoir (such as the embodiment of FIG. 6), the user experience differences between a full API concentration dose form and a reduced API concentration dose form can be masked by adding flavoring and/or anesthetic (for API's like diphenhydramine that have a topical anesthetic effect) so that the user thinks they are receiving the full strength API dose form when they receive the reduced concentration dose form.

In embodiments in which the API is a sleep aid, when the user awakes, the device or associated smartphone prompts the user to enter their assessment of how quickly they fell asleep and how rested they feel in the morning and uses this information to further modify the personalized API dispensing.

Also in embodiments in which the API is a sleep aid, the device can further interact with other sleep monitoring devices such as motion activated devices such as a wrist band, heart rate or heart rate variability monitors, under mattress monitors, EEG monitors, or other devices to ascertain sleep onset and quality and adjust dosage accordingly.

For example, if the user is having a hard time falling to sleep but their schedule indicates that they can sleep for 8 hours, the device when requested will deliver a higher dose such as 25-50 mg sufficient to initiate sleep in the user. This dose as well as others per the examples below, are adjusted automatically based on machine learning from similar historical requests made by the user.

For example, if a user wakes up after 3-4 hours of sleep, a common problem in older adults and had earlier requested a dose which was delivered as 10 mg, and if the user reports in the morning that they were not able to fall to sleep again (or such information was derived from the user's connected sleep monitoring devices), the dose would automatically be increased to 15-20 mg next time a similar request was made based on the device's machine learning algorithm.

In another example, if the user is travelling through multiple time zones and requests a dose to initiate sleep at a time their circadian clock does not support sleep initiation, a 25 mg dose would be offered to initiate sleep. If the user wakes up after a few hours and wishes to complete a longer sleep cycle of 8 hours, another dose of 10-15 mg would be offered.

The device's access to the history of API doses dispensed under various conditions (such as those described above) together with user self-reporting or automatic capture of the dose efficacy allows the system to personalize and minimize API doses for each scenario and insures that no overdosing will be possible, thus increasing efficacy of the API while reducing its side effects.

In yet another aspect of the invention this system can be used to dispense APIs such as Adderall (amphetamine), Ritalin (methylphenidate), Concerta (methylphenidate), Focalin, dexmethylphenidate), Daytrana (methylphenidate patch), Metadate (methylphenidate), Methylin (methylphenidate), Dexedrine (dextroamphetamine), to treat ADD in children and adults where an app used by the user or others (such as a parent or a teacher) may be used to rate the daily effect on the user's ADD condition by rating one or several aspects of the user's behavior. Conversely, the rating of the child behavior from one or multiple observers could be aggregated and analyzed, and, via the smart dispenser, may prompt the child to take a just-in-time dose of medicine tailored to minimize drug exposure and tolerance yet provide the full efficacy.

In yet another aspect of the invention this system can be used to dispense APIs such as SSRI's including fluoxetine (Prozac, Selfemra), paroxetine (Paxil, Pexeva), sertraline (Zoloft), citalopram (Celexa) and escitalopram (Lexapro) or serotonin and norepinephrine reuptake inhibitors (SNRIs) to treat depression where an app used by the user or others such as a parent or others to rate the user's psychological status and thus adjust the amount of API dispensed.

In yet another aspect of the invention, this system can be used to dispense APIs to treat diabetes (metformin), hypothyrodism (levothyroxine), anti-epileptics (carbmazepine), congestive heart failure (digoxin), chronic obstructive pulmonary disease (theophylline), Parkinson disease (levodopa and carbidopa in a fixed ratio from one cartridge or in varying ratios from dual cartridge system). In these cases, doses might be individualized by the physician with patients using a communication device such as their smart phone to document efficacy and side effects which could be subsequently analyzed by the physician or by automatic cloud based algorithms for possible adjustments in dosing.

In yet another aspect of the invention this system can be used to dispense APIs such as phentermine to depress the user's appetite where an app may be used by the user to rate their hunger and enter their weight manually or automatically from a scale in order to achieve a desired weight loss.

In yet another aspect of the invention, this system can be used to dispense APIs such as NSAIDs or opioids to treat pain where an app may be used to prompt the user to enter their level of pain while monitoring response to previous doses as well as maximum total dose delivered to ensure that the user does not accidentally overdose.

In yet another aspect of the invention, this system can be used to assess the user's condition. The pattern of use of the smart dispenser analyzed by a cloud analytic platform can prompt a remote clinician to intervene. For example, bouts of insomnia, depression, or pain as indicated by the pattern of use of the smart dispenser, can prompt changing remotely the maximum dose or other dispensing parameters for a particular user or remotely initiating a different therapeutic intervention.

In yet another aspect of the invention an algorithm of randomly alternating active with sub-therapeutic or placebo doses are used as probes to continually derive a minimally efficacious dose.

In yet another aspect of the invention, the use pattern of the dispenser is communicated to a cloud-based system allowing for automatic monitoring of the patient's compliance with taking the medication. Alerts can be generated automatically for follow up by healthcare professionals or by app on a smartphone.

In yet another aspect of the invention, a healthcare professional can remotely modify the dosing algorithm of the dispenser. The new instructions can be hidden from the patient or communicated to the patient depending on the desired placebo effect.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” or “substantially” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others.

Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-26. (canceled)

27. A method of administering a sleep aid from a dispensing system to a user, the method comprising:
actuating a user input of the dispensing system at a first time;
in response to actuation of the user input at the first time, dispensing a first volume of a dose form into the user's oral cavity, the dose form comprising an amount of the sleep aid;
masking from the user the amount of the sleep aid in the first volume;
obtaining information regarding effectiveness of the dispensing of the first volume in aiding the user's sleep;
actuating the user input of the dispensing system a second time after the first time to dispense a second volume of the dose form into the user's oral cavity, the second volume being automatically controlled by the dispensing system to differ from the first volume based on the information and;
masking from the user the amount of the sleep aid in the second volume.

28. The method of claim 27 wherein the sleep aid is diphenhydramine.

29. The method of claim 27 wherein each of the masking steps comprises dispensing a detectable ingredient with the sleep aid in the first volume of the dose form.

30. The method of claim 29 wherein the detectable ingredient comprises a flavoring agent.

31. The method of claim 29 wherein the detectable ingredient is a numbing agent.

32. The method of claim 27 wherein the second volume is less than the first volume.

33. The method of claim 32 wherein the second volume comprises a sub-therapeutic dose of the sleep aid.

34. The method of claim 32 further comprising obtaining information about expected hours of remaining sleep time, the second volume being automatically controlled by the dispensing system to differ from the first volume based on the expected hours of remaining sleep time.

35. The method of claim 27 wherein the second volume is more than the first volume.

36. The method of claim 27 wherein the information comprises quality of the user's sleep.

37. The method of claim 36 wherein the information comprises length of user's sleep.

38. The method of claim 36 wherein the information comprises the user's feeling of restedness.

39. The method of claim 27 further comprising, before dispensing the second volume, determining whether the total dose delivered in the first volume is less than a predetermined maximum dose and, if not, providing an indication to the user that the second volume of the dose form will not be delivered.

40. The method of claim 27 further comprising, before dispensing the first volume, determining whether the user is an authorized user.

41. The method of claim 27 wherein the dispensing system comprises a handheld dispenser.

42. The method of claim 41 wherein the dispensing system comprises a replaceable cartridge within the handheld dispenser and containing the dose form.

43. The method of claim 42 wherein the cartridge comprises a dispensing tip configured to be inserted into the user's mouth, the method further comprising inserting the dispensing tip into the user's mouth prior to dispensing the first volume or the second volume.

44. The method of claim 42 wherein the dispenser further comprises a window through which contents of the cartridge can be viewed.

45. The method of claim 42 wherein the replaceable cartridge is a first replaceable cartridge and the dose form is a first dose form, the dispensing system further comprising a second replaceable cartridge within the handheld dispenser and containing a second dose form different from the first dose form.

46. The method of claim 45 wherein the dispensing system further comprises a dispensing tip in communication with the first replaceable cartridge and the second replaceable cartridge, the method further comprising inserting the dispensing tip into the user's mouth prior to dispensing the first volume or the second volume.

47. The method of claim 27 wherein the dispensing system further comprises a smartphone running an application, the method further comprising entering the information through the application on the smartphone.

48. The method of claim 47 wherein the method further comprises setting the second volume with the application on the smartphone and communicating information about the second volume from the smartphone to the dispenser.

49. The method of claim 27 wherein the sleep aid dose in the first volume is substantially absorbed by oral mucosa of the user.

50. The method of claim 27 wherein the first volume is small enough to avoid stimulation of a swallowing instinct of the user.

51. The method of claim 27 wherein the dose form further comprises a penetration enhancer.

52. The method of claim 27 wherein the dose form further comprises a viscosity modifier.

53.-72. (canceled)

73. The method of claim 27 wherein each of the masking steps comprises providing a dispensing cue that masks the first volume and the second volume.

74. The method of claim 73 wherein the dispensing cue comprises running a motor of the dispensing system longer than is necessary to dispense the first volume or the second volume.

75. The method of claim 73 wherein the dispensing cue comprises flashing lights of the dispensing system longer than a delivery period of the first volume or the second volume.
